(19) United States
(12) Patent Application Publication
(10) Pub. No.: US 2010/0284979 A1
(43) Pub. Date: Nov. 11, 2010

(54) Modulation of Tissue Fatty Acid Composition of a Host by Human Gut Bacteria

(75) Inventors: Liam O'Mahony, Rathcormac (IE); Fergus Shanahan, Kinsale (IE); Eamonn Quigley, Kinsale (IE); Paul Ross, Kilworth (IE); Catherine Stanton, Kilworth (IE)

Correspondence Address: FOLEY & LARDNER LLP, 975 PAGE MILL ROAD, PALO ALTO, CA, 94304, US

(73) Assignees: UNIVERSITY COLLEGE CORK, NATIONAL UNIVERSITY OF IRELAND, CORK, Cork (IE); TEAGASC, The Agriculture and Food Development Authority, Dublin (IE); ALIMENTARY HEALTH LTD., Cork (IE)

(21) Appl. No.: 12/681,045

(22) PCT Filed: Sep. 30, 2008
(86) PCT No.: PCT/EP2008/063106

§ 371 Date: Jul. 13, 2010

(30) Foreign Application Priority Data
Oct. 1, 2007 (IE) 2007/0704
Dec. 7, 2007 (IE) 2007/0895

(51) Int. Cl. A61K 35/74; A61P 25/28; A61P 19/02; A61P 25/24; A61P 17/00; A61P 11/06; C12N 1/20

(52) U.S. Cl. 424/93.44; 424/93.4; 424/93.45; 435/252.1

(57) ABSTRACT

The current invention provides use of a CLA-producing bacterium for the in vivo conversion in the gut of polyunsaturated fatty acids to CLA. The CLA-producing bacterium is selected from one or more of the group consisting of propionibacteria, lactobacilli, lactococci and streptococci, and bifidobacteria.

### FIELD OF THE INVENTION

The present invention relates to the use of microbial species to modulate the tissue fatty acid composition of a host using human gut bacteria and to convert polyunsaturated fatty acids to CLA in vivo. The invention provides methods and compositions for use in such methods.

### BACKGROUND TO THE INVENTION

The human body can produce all but two of the fatty acids it requires; thus linoleic acid (C18:2n-6, precursor of n-6 series of fatty acids) and α-linolenic acid (C18:3n-3, precursor of n-3 series of fatty acids) are essential dietary fatty acids. Although mammalian cells cannot synthesize these fatty acids, they can metabolize them into more physiologically active compounds through a series of elongation and desaturation reactions, in which linoleic acid is converted to arachidonic acid (C20:4n-6) and α-linolenic acid is metabolized to EPA (C20:5n-3) via the action of Δ⁶ desaturase, Δ⁵ desaturase and elongase enzymes (1). The resulting highly unsaturated fatty acid metabolites are necessary for the functioning of the cell membrane, the proper development and functioning of the brain and nervous systems, and the production of inflammatory mediators, i.e. eicosanoids (thromboxanes, leukotrienes and prostaglandins) (1, 2). The eicosanoids derived from arachidonic acid, such as the 2-series prostaglandins and the 4-series leukotrienes, are in general being ascribed to be proinflammatory and to exhibit disease-propagating effects if present in abundance (3), whereas the eicosanoids derived from EPA, such as the 3-series prostaglandins and the 5-series leukotrienes are considered to be less inflammatory or even anti-inflammatory (3, 4). Thus, increasing the ratio of n-3 to n-6 fatty acids in the diet, and consequently favouring the production of EPA, the balance of eicosanoids can be shifted towards a less inflammatory mixture. Conjugated linoleic acid (CLA) refers to a group of polyunsaturated fatty acids which are positional and geometric isomers of linoleic acid [C18:2 cis-9 (c9), cis-12 (c12) octadecadienoic acid]. CLA is a natural component of milk fat due to the microbial biohydrogenation of linoleic acid in the rumen. CLA is thus found in the milk fat and meat of ruminant animals. The predominant CLA isomer found in nature, and in food, is the c9, t11 CLA isomer. It has been proposed that CLA has positive effects on many aspects of human health. Most notably are the effects within the areas of cancer, immune modulation, atherosclerosis and obesity. The mechanisms of action underlying these biological properties are not clearly understood however. CLA was first implicated in down-regulating the generation of inducible eicosanoids (i.e. PGE₂ and LTB₄) involved in early micro-inflammation events, but more recently, CLA has also been shown to modulate the expression of genes regulated by peroxisome proliferator-activated receptors (PPARs). PPARs (α, β/δ, and γ) are ligand-activated transcription factors that increase transcription of target genes by binding to a specific nucleotide sequence in the gene's promoter. Numerous works have shown CLA's anti-inflammatory effect in the gastrointestinal tract, an effect linked to its ability to interfere with proinflammatory intracellular signalling cascades. The human gut harbours a diverse bacterial community that can comprise more than 1000 different species, out-numbering the human somatic and germ cells 10-fold (5). There is now compelling evidence that the enteric microbiota plays an important role in the health and well-being of the host. For example, evidence obtained from comparative studies of germ-free and conventionally colonised animals have shown that the human enteric microbiota exerts a conditioning effect on intestinal homeostasis, delivering regulatory signals to the epithelium and instructing mucosal immune responses (5, 6). Furthermore, the enteric microbiota was recently established as a regulator of fat storage (7). Little is known regarding the interplay between members of the human enteric microbiota and fatty acids. However, there are some indications that intestinal bacteria within the gastrointestinal tract (GIT) may interact with different fatty acids. For example, it has been shown that administration of probiotics (Lactobacillus rhamnosus GG and Bifidobacterium animalis subsp. lactis Bb12) to pregnant women affected placental fatty acid composition (8). Moreover, Kankaanpää et al. (9) demonstrated that administration of formula supplemented with different probiotics (B. animalis subsp. lactis Bb12 and L. rhamnosus GG) to infants resulted in changes in the fatty acid composition of serum lipids. Recent studies have also reported that intestinal bacteria of human origin can convert dietary linoleic acid to bioactive isomers of CLA both in vitro and in vivo (10-13). Some bacteria of marine origin even possess the metabolic capacity to synthesize EPA and DHA (14-16). In these bacteria, EPA and DHA are synthesized de novo by polyunsaturated fatty acid synthase genes rather than by chain elongation and desaturation of existing fatty acids (17, 18). The current inventors have shown that increased concentrations of EPA and DHA were obtained in adipose tissue of mice administered the metabolically active strain B. breve NCIMB 702258 compared to unsupplemented mice (unpublished data). It has been demonstrated that bacterial cultures, other than rumen bacteria, possess the ability to generate c9, t11 CLA from free linoleic acid. These include the intestinal microflora of rats, propionibacteria, lactobacilli, lactococci and streptococci, and bifidobacteria, including a number of strains of human origin. Of particular interest are bifidobacteria, with some clinical studies linking their presence in the gut with specific health effects, including improvement of gastrointestinal disturbances, enhancement of immune function, and cancer suppression. Unlike ruminants, human production of CLA from linoleic acid does not appear to occur at any significant level. The amount of CLA in human adipose tissue is thought to be directly related to dietary intake. The best source of CLA is fat from ruminants, however since consumption of fat from ruminants is usually not recommended by nutritionists (due to its high concentration of saturated fatty acids) the ingestion of a CLA-producing bacteria could be an option for maintaining and supplementing levels of CLA in the gut. CLA has been shown to be generated in vitro from linoleic acid, but according to Bassaganya-Riera et al. (16) and Kamlage et al. (21), this synthesis appears to be inhibited in vivo.

Certain metabolic capabilities of microorganisms that easily are observed in vitro do not necessarily occur in vivo. This appears to be true for enzymatic activities that are not essential for survival of the microorganism such as the biotransformation of non-nutritive dietary compounds such as LA. CLA production has not been described as a mechanism by which probiotics exert anti-inflammatory effects. Several studies have investigated the bioproduction of CLA by various lactobacilli and bifidobacteria but the action of this CLA has not been investigated. Inflammatory bowel disease (IBD) is a widespread and debilitating illness afflicting over 3.5 million people worldwide. It is a chronic disease of the digestive tract, and usually refers to two related conditions of unknown cause, ulcerative colitis and Crohn's disease, which are characterized by chronic and spontaneously relapsing inflammation leading to destruction of the gut mucosa. These two diseases are important since they are increasing in frequency, disabling for many patients, and generating a significant burden on the health care system. Although the etiology of IBD remains unknown, there is increasing experimental evidence to support a role for luminal bacteria in the initiation and progression of these intestinal conditions; probably related to an imbalance in the intestinal microflora, relative predominance of aggressive bacteria and insufficient amount of protective species. A lot of data supports the theory that these diseases represent the outcome of 3 essential interactive factors: host susceptibility, enteric microflora, and mucosal immunity.

Current treatments for IBD include corticosteroids, antibiotics and immunomodulators. Although IBD therapies have improved, they still are only modestly successful for the long-term management of the disease and result in significant side effects. Therefore, exploring novel preventive or therapeutic interventions remains important. A possible therapeutic approach in IBD therapy is the administration of probiotic microorganisms. Probiotics are defined as live microorganisms that confer health benefits to the human host through a number of mechanisms. Probiotic bacteria are attractive alternatives for the treatment of gastrointestinal inflammation due to their effects on the composition of the gut flora and activity on the immune system. Recently, some investigators have reported success with different strains of probiotics in the treatment of chronic intestinal diseases such as ulcerative colitis (5), Crohn's disease (6), and pouchitis (7). E. coli (Nissle 1917), the yeast Saccharomyces boulardii, Lactobacillus GG, and VSL# 3, a cocktail of eight different strains, have been used successfully in human pathology. In addition, indirect evidence demonstrates the potential impact of nutrition in general and lipid nutrition in particular in modulating the course of IBD. For example, in a study by Hontecillas et al. (10), conjugated linoleic acid (CLA), a dietary fatty acid, proved to ameliorate IBD in a pig model of bacterial-induced colitis. CLA ameliorated intestinal lesion development, prevented growth suppression and maintained or induced PPAR γ expression while repressing IFN-γ expression. N-3 polyunsaturated fatty acids (PUFA) [i.e., docosahexaenoic (DHA) and eicosapentaenoic (EPA)] are other beneficial fatty acids that elicit potent anti-inflammatory and immunoregulatory properties (11). In similar action to dietary CLA, n-3 PUFA have been reported to ameliorate intestinal inflammation in animal models of IBD (9).

### OBJECT OF THE INVENTION

It is an object of the invention to provide compositions and methods, which can lead to the generation of health-promoting fatty acids within the mammalian body. A further object is to provide compositions and methods, which reduce inflammation in the digestive tract. The methods and compositions may reduce or alleviate the symptoms of inflammatory bowel disease. A still further objective is to provide probiotic compositions having the above effects, which can easily be consumed. The probiotic compositions may be foodstuffs or pharmaceutical products. A still further object is to provide methods for the in vivo conversion in the gut of linoleic acid to CLA and methods to alter the fatty acid composition of internal organs of the body. A further object was to provide co-administration of commensal bifidobacteria, with ability to produce bioactive isomers of conjugated linoleic acid (CLA) in combination with α-linolenic acid influence the EPA and DHA concentrations of different tissues.

### SUMMARY OF THE INVENTION

According to the present invention there is provided use of a CLA-producing bacterium in the preparation of a composition for the treatment of Inflammatory Disease. The CLA-producing bacterium may be selected from the group consisting of propionibacteria, lactobacilli, lactococci and streptococci, and bifidobacteria. The bacterium may be Bif. Breve, Bif. Lactis, Bif. Dentum, Lactobacillus rhamnosus or Butyrivibrio fibrisolvens. In particular, the CLA-producing bacterium may be the publicly available strain Bifidobacterium breve as deposited at the National Culture of Industrial and Marine Bacteria under the accession no. 702258 or B. breve DPC 6330 as deposited at the National Culture of Industrial and Marine Bacteria under the accession no. 41497 on 28 Sep. 2007, or B. longum DPC 6315 as deposited at the National Culture of Industrial and Marine Bacteria under the accession no. 41508 on 18 Oct. 2007.

The invention also provides use of a CLA—producing bacterium for the in vivo conversion in the gut of polyunsaturated fatty acids, such as linoleic acid to CLA. The invention also provides use of a CLA—producing bacterium to alter the fatty acid composition of internal organs of the body. The CLA-producing bacterium may be as defined above.

A further aspect the invention relates to a probiotic composition comprising a CLA producing organism together with pharmaceutically acceptable or nutritionally acceptable additives. The CLA-producing bacterium may be as defined above. The probiotic composition may be a pharmaceutical composition or a foodstuff composition. In the case of a pharmaceutical composition, it may be formulated as a tablet, capsule, suspension, powder of the like, and contain pharmaceutically acceptable carriers or excipents, as would be well known in the art. If formulated as a foodstuff, the composition may be a yoghurt, a yoghurt drink, a cheese, a milk, a spread, a fruit juice, a water which is either flavoured or unflavoured or any other edible composition. This probiotic combination may lead to a more healthy/desirable fatty acid composition of host tissues such as the liver where it may protect against non-alcohol—induced fatty liver disease. The probiotic compositions can reduce gut inflammation in diseases such as Inflammatory Bowel Syndrome or Inflammatory Bowel Disease, rheumatoid arthritis, multiple sclerosis, Alzheimer's disease, eczema, asthma or psychiatric diseases such as depression. The composition may also be formulated as an animal feedstuff, together with conventional animal feed ingredients. The probiotic composition may further comprise a substrate, which can be converted into a bioactive compound in vivo by the CLA producing organism. The substrate may be a polyunsaturated fatty acid, such as, but not limited to linoleic acid, linolenic acid, oleic acid, palmitic acid, or stearic acid. In a still further aspect the invention provides a method of converting dietary polyunsaturated fatty acids, such as linoleic acid to CLA in vivo comprising administration to a subject of a live CLA-producing bacterial strain. The invention also provides a method of altering the fatty acid composition of internal organs of the body comprising administering to a subject a live CLA-producing bacterial strain. Suitable bacterial strains are as defined above. The invention also provides CLA producing strains isolated from the human intestine, B. breve DPC 6330, which has highest conversion rate of 76.65+/−1.75% conversion of linoleic acid to c9, t11 CLA, and B. breve DPC 6331, which has 60.12+/−5.14% conversion, compared to the publicly available strain B. breve NCIMB 702258 which has a conversion rate to c9, t11 CLA of 60%.

In a still further aspect the invention provides a method to drive DHA (docosahexaenoic acid)/EPA (eicosapentaenoic acid) incorporation into host tissues using dietary CLA or a strain producing CLA, in order to improve memory loss and cognition.

### BRIEF DESCRIPTION OF THE DRAWINGS

FIG. 1. Cytokine production by stimulated splenocytes. A-C shows cytokine production following stimulation with antiCD3-antiCD28 monoclonal antibodies, D-E shows cytokine production following stimulation with the proinflammatory bacterium S. typhimurium UK1. There was a significant difference in the proinflammatory cytokines IFN-γ, TNF-α, and IL-6 in the groups fed c9,t11 CLA (group 4) and the probiotic B. breve NCIMB 702258 (group 3) (*p<0.05). Results are expressed as mean cytokine levels±standard error per group (n=8).

FIG. 2. Body weight development. Values are means±SEM (n=9). FIG. 3. Recovery of B. breve NCIMB 702258 in stool from probiotic fed mice (group A). Results are expressed as log mean colony forming units (CFU/g)±SD. No probiotics were isolated from mice in the placebo group.

FIG. 4. Bioproduction of c9, t11 CLA by B. breve NCIMB 702258 occurs in vivo. Group A shows mice fed LA together with B. breve NCIMB 702258, group B shows LA-fed mice. Columns with * are statistically significant different from corresponding columns in control group (B).

FIG. 5. c9, t11 CLA incorporation into faeces harvested after 8 weeks feeding. Group A had a 2.4-fold higher amount of c9, t11 CLA in the faeces compared to group B (p=0.001). A high level of c9, t11 CLA in the faeces correlated with a low level of LA (r=−0.863).

FIG. 6. Enumerated B. breve NCIMB 702258 in the large intestinal contents. B. breve NCIMB 702258 was detected in the large intestine at ˜4.6×10⁵ CFU/g in mice that received B. breve in combination with α-linolenic acid (group A) and ˜1.4×10⁶ CFU/g in the mice that received B. breve without α-linolenic acid (group C) (p>0.05). B. breve NCIMB 702258 was not isolated from any of the mice that did not receive B. breve (group B and group D). Enumeration of lactobacilli was performed using lactobacilli selective agar (LBS). The numbers of CFU obtained on LBS did not differ between the groups (p>0.05). Results are expressed as log means CFU±SEM (CFU/g).

FIG. 7. Incorporation of EPA in liver, adipose tissue and brain demonstrating that dietary supplementation with B. breve in combination with α-linolenic acid (group A) increases the content of EPA in the liver significantly compared to mice receiving α-linolenic acid without the B. breve strain (group B) (p<0.05). ^{A, B, C, D} Different superscript numbers within a column indicate significant differences (n=8, p<0.05). EPA is expressed as Mean±SEM g/100 g FAME. FIG. 8. Incorporation of DHA in liver, adipose tissue and brain demonstrating that dietary supplementation with B. breve in combination with α-linolenic acid (group A) increases the content of DHA in the brain significantly compared to mice receiving α-linolenic acid without the B. breve strain (group B) (p<0.05). ^{A, B, C, D} Different superscript numbers within a column indicate significant differences (n=8, p<0.05). DHA is expressed as Mean±SEM g/100 g FAME.

### DETAILED DESCRIPTION OF THE DRAWINGS

### Materials and Methods

Severe Combined Immuno Deficient (SCID) mice were used in this study, which had been induced with colitis through adoptive transfer of splenic CD4⁺ CD45RB^{high} T cells from BALB/c mice. A group of mice (n=8) were fed a linoleic acid (LA) supplemented diet (1%), a second group received pure cis-9, trans-11 CLA (1%), a third group received LA (1%) together with the CLA-producing strain B. breve NCIMB 702258 (a daily dose of 10⁹ organisms), a fourth group were fed a standard diet together with B. breve NCIMB 702258 (a daily dose of 10⁹ organisms), and a fifth group were fed only the standard diet. Ten weeks after colitis induction, cytokine production by splenocytes was measured in vitro by ELISA, myeloperoxidase (MPO) activity was determined in colonic homogenates and fatty acid composition in liver, adipose tissue, cecum and colon was determined by gas liquid chromatography (GLC). Similar studies have also been conducted in pigs (data not shown here) and similar results obtained.

### Animals and Experimental Design

SCID mice were purchased from Harlan ltd. (Briester, Oxon, UK) at 6 weeks of age and fed a normal diet for 1 week to stabilize all metabolic conditions. Each cage contained one mouse. Mice were exposed to a 12-h light:dark cycle and maintained at a constant temperature of 25° C. One week after arrival, mice were divided into five groups for different dietary treatments (Table I). For linoleic acid and c9t11 CLA treatment, a powdered diet blended with the drug was administered for 10 weeks to yield a dose of drug at approximately 90 mg/day/mouse (this is based on preliminary experiments by Bassaganya-Riera et al. (22) that established an optimal dose of fatty acids of 1 g/100 g of diet).

Male BALB/c mice were purchased from Harlan Ltd. (Briester, Oxon, UK). One week after arrival, these mice were divided into two groups (n=9) for different dietary treatments. Group A received 1% LA in their diet together with approximately 1×10⁹ live B. breve NCIMB 702258 per day. Group B received 1% LA in their diet. For LA treatment, a powdered diet blended with the drug was administered for 8 weeks to yield a dose of drug at approximately 90 mg/day/mouse (this is based on preliminary experiments by Bassaganya-Riera et al. (1) that established an optimal dose of fatty acids of 1 g/100 g). Animals were fed standard mouse chow ad libitum with free access to water at all times. The ingredients and composition of the basal diet was as follows: Ingredients; Soyabean extracted toasted, Cane molasses, Sunflower seed extracted, Wheat, Barley, Mineral/vitamin, Soya(bean) hulls, Maize gluten, Calcium carbonate (limestone flour), Sodium chloride.

### Composition;

### Protein 17.0%

Oil 3.0% (background oil, no oil is added. 1.6% comes from wheatfeed, 0.9% from oats, 0.2% from sunflower)

### Fibre 8.50%

### Ash 7.80%

### Moisture 14.0%

Copper 30.00 mg/kg
Vitamin A 13500 iu/kg
Vitamin D 3000 iu/kg
Vitamin E 90 iu/kg

Of the total fatty acids that are in the diet, Linoleic acid is responsible for approximately 50% (Oleic acid 22%, palmitic acid 17.5%, linolenic acid 3.7% and stearic acid 2.8%). The amount of oil in their diet is 3%, which comes from wheatfeed, soyabean, oats and sunflower.

After 8 weeks on experimental diets, the mice were sacrificed by cervical dislocation. Livers, hearts, colons, small intestines and cecums were removed from the carcasses, blotted dry on filter paper, weighed and frozen in liquid nitrogen. All samples were stored at −80° C. until processed.

### Induction of Colitis

SCID mice were induced with colitis through adoptive transfer of the CD4⁺ CD45RB^{high} subpopulation of CD4+αT cells from normal BALB/c mice (Mackay et al. 1998).

### Probiotic Strain

B. breve National Culture for Industrial and Marine Bacteria deposit no. 702258 was originally isolated from an infant intestine and has previously been shown to be resistant to intestinal acid and bile and to adhere to human intestinal cells (unpublished data). This strain has previously been shown to be an efficient CLA producer, converting up to 65% LA to c9, t11 CLA when grown in 0.55 mg ml⁻¹ LA in vitro (23).

A spontaneous rifampicin resistant variant of the strain was isolated, prior to initiation of this study, in order to facilitate uncomplicated identification of this bacterium from all other bifidobacteria.

### Preparation and Administration of the Probiotic

B. breve NCIMB 702258 was initially grown in modified MRS medium (mMRS) supplemented with 0.05% (wt/vol)_{L}-cysteine hydrochloride (98% pure; Sigma Chemical Co., St. Louis, Mo.) by incubating overnight at 37° C. under anaerobic conditions. The culture was pelleted by centrifugation (7000 g, 15 min, 4° C.). The pellet was washed twice in PBS (Sigma) and then resuspended at 1×10¹° cells/ml in 15% trehalose (Sigma). 1 ml was aliquoted into 2-ml vials and freezedried using a 24 hour programme (freeze temp −40° C., condenser set point −60, vacuum set point 600 m Torr). Each mouse consumed approximately 1×10⁹ live microorganisms per day. This was achieved by resuspending appropriate quantities of freeze-dried powder in the water that the mice consumed ad libitum.

### Assessment of Colitis

After the adoptive transfer, mice were weighed three times a week and examined for clinical signs of disease associated with colitis (ie. rectal bleeding, diarrhoea and rectal prolapse). After 10 weeks on experimental diets, the mice were sacrificed by cervical dislocation. Livers, adipose tissue, spleens, colons and cecums were removed from the carcasses, blotted dry on filter paper, weighed and frozen in liquid nitrogen. All samples were stored at −80° C. until processed. The colon was divided into different sections for MPO activity and fatty acid composition.

### Myeloperoxidase (MPO) Assay

Myeloperoxidase activity was measured according to the method by Krawisz et al. (24). Samples were excised from each animal and rapidly rinsed with ice-cold PBS, blotted dry and frozen at −80° C. The tissue was thawed and homogenized in 0.5 ml PBS. 200 μl of each homogenised sample was transferred to separate eppendorf tubes and 400 μl of 0.5% hexadecyltrimethylammonium bromide (HTAB) solution (Sigma, St Louis, Mo., USA) was added. After vortexing for 2 min, samples were spinned at 5.000 rpm for 5 min after which the supernatant was collected. Using a 96 well microtitre plate, 50 μl of each sample was added to duplicate wells. 12.5 μl of hydrogen peroxide (H₂O₂, 30% (w/v), Sigma) was added to each well followed by 200 μl of O-dianisidine reaction solution (Sigma). The absorbance (OD) was measured spectrophotometrically at 450 nm at 1 min intervals for 15 min. The total soluble protein concentration in the samples was estimated using the method by Bradford et al. (48). This was performed using a Bio-Rad protein assay dye reagent kit (Bio-Rad Laboratories, Hercules, Calif.). Results are expressed as U/mg protein.

### Cytokine Production by Splenocytes.

Cytokine production in response to defined stimuli, in vitro, was measured using enzyme-linked immunosorbent assay (ELISA). Splenocyte isolation was performed using an erythrocyte lysing kit (R&D Systems). The spleens of all mice were removed at the time of sacrifice, blotted on filter paper and weighed. Each spleen was immediately placed in Hanks' buffer containing 10% fetal calf serum. The spleen was teased apart and sieved through a cell strainer into a 50 ml centrifuge tube. Cells were centrifuged for 10 minutes at 200 g. The cells were resuspended in 2 ml M-lyse buffer, which lyses red blood cells, and incubated for 8 minutes at room temperature. Lysis was deactivated using wash buffer and the cell suspension was centrifuged for 10 minutes at 200 g. Cells were resuspended in DMEM (10% fetal calf serum, 1% Pen/Strep, Sigma) and diluted to 1×10⁶ cells/ml for in vitro culturing. The isolated lymphocytes were cocultured with the proinflammatory bacterium Salmonella typhimurium UK1 (1×10⁶ cells/ml) and with antiCD3-antiCD28 monoclonal antibodies for 48 hours at 37° C. Cell supernatants were isolated and stored at −80° C. Cytokine analysis was performed on the supernatants using a BD™ Cytometric Bead Array (CBA) Mouse Inflammation Kit (BD Biosciences, US).

### Microbial Analysis

Fresh faecal samples were taken directly from the anus of every mouse every second week for microbial analysis and fatty acid analysis. Microbial analysis of the samples involved enumeration of B. breve NCIMB 702258. This analysis was performed by pour plating onto MRS agar supplemented with 0.05% (wt/vol)_{L}-cysteine hydrochloride (98% pure; Sigma Chemical Co., St. Louis, Mo., USA), 100 μg of Mupirocin (Oxoid)/ml (4) and rifampicin (Sigma). Large intestinal contents were also sampled at sacrifice for enumeration of the administered B. breve strain and for enumeration on Lactobacillus selective agar (LBS) (Becton Dickinson Co, Cockeysville, USA). Microbial analysis of B. breve NCIMB 702258 was performed by pour plating onto mMRS agar supplemented with 100 μg of mupirocin (Oxoid)/ml (Rada, 1997) and 100 μg rifampicin (Sigma)/ml. Agar plates were incubated anaerobically for 72 hrs at 37° C. Anaerobic environments were created using CO₂ generating kits (Anaerocult A; Merck, Darmstadt, Germany) in sealed gas jars.

### Lipid Extraction and Fatty Acid Analysis

Lipids were extracted with Chloroform:Methanol 2:1 v/v according to Folch et al. (25). Briefly, tissue or faecal samples, ˜1 g liver, 300 mg small intestine, 200 mg adipose tissue, 250 mg colon, 800 mg cecum or 100 mg feaces were homogenised in over a 25 fold excess of CHCl₃:CH₃OH (2:1 v/v) and washed with 0.88% KCl solution. Excess solvent was dried down under a gentle stream of N₂ at 40° C. and lipids were stored at −20° C. in 1 ml chloroform. Fatty acid methyl esters (FAME) were prepared using first 10 ml 0.5N NaOH in methanol for 10 min at 90° C. followed by 10 ml 14% BF₃ in methanol for 10 min at 90° C. (26). FAME was recovered with hexane. Prior to GC analysis samples were dried over 0.5 g of anhydrous sodium sulphate for an hour and stored at −20° C. FAME were separated by gas liquid chromatography (GLC Varian 3400, Varian, Walnut Creek, Calif. USA fitted with a flame ionization detector) using a Chrompack CP Sil 88 column (Chrompack, Middleton, The Netherlands, 100 m×0.25 mm i.d., 0.20 μm film thickness) and He as a carrier gas. The column oven was programmed to be held initially at 80° C. for 8 minutes then increased 8.5° C./min to a final column temperature of 200° C. The injection volume used was 0.6 μl with automatic sample injection with a splitless on SPI on-column temperature programmable injector. Data was recorded and analysed on a Minichrom PC system (VG Data System, Manchester, U.K.). Tri-heptadecanoate (Sigma) was used as an internal standard, and peaks were identified with reference to retention times of fatty acids in a standard mixture. All fatty acid results are shown as mean±SD (n=8)g/100 g FAME.

### Statistical Analysis

Data are expressed as the mean value per group of mice±standard deviation.

Data was analysed by MINITAB® Release 14 statistical software, Lead Technologies, Inc. and data were tested as appropriate by ANOVA or Kruskal-Wallis (27) tests in order to assess if differences between groups are significant. A P-value of <0.05 was considered to be statistically significant.

Preparation and Administration of B. breve NCIMB 702258

Rifampicin resistant variants of the B. breve strain were isolated by spread-plating ˜10⁹ colony forming units (CFU) from an overnight culture onto MRS agar (de Man, Rogosa & Sharpe) (Difco Laboratories, Detroit, Mich., USA) supplemented with 0.05% (w/v) L-cysteine hydrochloride (98% pure; Sigma Chemical Co., St. Louis, Mo.) (mMRS) containing 500 μg/ml rifampicin (Sigma Chemical Co., Poole, Dorset, UK). Following anaerobic incubation at 37° C. for 3 days, colonies were stocked in mMRS broth containing 40% (v/v) glycerol and stored at −80° C. To confirm that the rifampicin resistant variant was identical to the parent strain, molecular fingerprinting using pulse-field gel electrophoresis (PFGE) was employed. Both parent and variant strains were routinely cultured at 37° C. in mMRS broth in anaerobic jars with CO₂-generating kits (Anaerocult A; Merck, Darmstadt, Germany). Importantly, the rifampicin resistant variant was comparable to the parent strain for CLA production.

Mice that did not receive the bacterial strain received placebo freeze-dried powder (15% w/v trehalose).

### Animals and Treatment

Female BALB/c mice were purchased from Harlan ltd. (Briester, Oxon, UK) at 8 weeks of age and fed a normal diet for 1 week to stabilize all metabolic conditions. The basal diet contained the following nutrient composition (w/w): nitrogen free extract (57.39%), crude protein (18.35%), moisture (10%), ash (6.27%), crude fibre (4.23%) and crude oil (3.36%), which consisted of saturated fatty acids: C12:0 (0.03%), C14:0 (0.14%), C16:0 (0.33%), C18:0 (0.06%), monounsaturated fatty acids: C14:1 (0.02%), C16:1 (10%), C18:1 (0.87%), polyunsaturated fatty acids: C18:2n-6 (0.96%), C18:3n-3 (0.11%), C20:4n-6 (0.11%). Mice were maintained at 4 per cage and exposed to a 12-h light:dark cycle at a constant temperature of 25° C. The mice were held at the Biological Services Unit in University College Cork. The animal experimentation was performed according to the guidelines for the care and use of laboratory animals approved by the Department of Health and Children.

One week after arrival, mice were divided into four groups (n=8/group) and subjected to the following dietary treatments: Group A were supplemented with 1% α-linolenic acid (w/w, triglyceride bound form, Larodan Fine Chemicals AB, Malmo, Sweden) in combination with approximately 1×10⁹ live B. breve NCIMB 702258 per mouse/day. Group B were supplemented with 1% α-linolenic acid and placebo freeze-dried powder, group C received standard diet supplemented with ˜1×10⁹ live B. breve NCIMB 702258, and group D received standard diet and placebo freeze-dried powder. Animals were fed standard mouse chow ad libitum with free access to water at all times. For α-linolenic acid treatment, a powdered standard diet was blended with the α-linolenic acid to yield a concentration of approximately 90 mg α-linolenic acid/day/mouse (based on Bassaganya-Riera et al. (19) who reported an optimal intake of fatty acids of 1 g/100 g). Following 8 weeks on experimental diets, the animals were sacrificed by cervical dislocation. Liver, adipose tissue and brain were removed from the carcasses, blotted dry on filter paper, weighed and frozen in liquid nitrogen. All samples were stored at −80° C. until processed.

### Microbial Analysis

Lipid extraction and fatty acid analysis Lipids were extracted according to the method by O'Fallon et al. (20). Briefly, samples were cut into 1.5-mm rectangular strips and placed into a screw-cap Pyrex culture tube together with 0.7 ml of 10 N KOH in water and 5.3 ml of MeOH. The tubes were incubated in a 55° C. water bath for 1.5 h with vigorous hand-shaking every 20 min. After cooling below room temperature, 0.58 ml of 24 NH₂SO₄ in water was added. The tubes were mixed by inversion and with precipitated K₂SO₄ present incubated again in 55° C. for 1.5 h with hand-shaking every 20 min. FAME were recovered by addition of 3 ml hexane and vortex mixed and separated by GLC (Varian 3400, Varian, Walnut Creek, Calif. USA fitted with a flame ionization detector) using a Chrompack CP Sil 88 column (Chrompack, Middleton, The Netherlands, 100 m×0.25 mm i.d., 0.20 μm film thickness) and He as carrier gas. The column oven was initially programmed at 80° C. for 8 min, and increased 8.5° C./min to a final column temperature of 200° C. The injection volume was 0.6 μl, with automatic sample injection on a SPI 1093 splitless on-column temperature programmable injector. Data were recorded and analysed on a Minichrom PC system (VG Data System, Manchester, U.K.). Peaks were identified with reference to retention times of fatty acids in a standard mixture. All fatty acid results are shown as mean±standard error mean (SEM) g/100 g FAME.

### Results

In this study, the ability of probiotic bacteria to exert anti-inflammatory effects through the production of CLA was assessed. The conversion of LA to c9, t11 CLA by B. breve NCIMB 702258 was investigated in vivo and also whether this bacterially produced CLA would have any beneficial effect on Inflammatory Bowel Disease (IBD). Throughout the study period, faecal recovery of B. breve NCIMB 702258 was confirmed in all probiotic fed mice but not in controls.

### Myeloperoxidase (MPO) Assay

Myeloperoxidase (MPO) activity, an index of quantitative inflammation and neutrophil infiltration in the mucosa was measured in colonic homogenates. As shown in Table II, the MPO activities were much higher in the placebo fed group than in other groups. The MPO activity was lowest in the mice fed c9, t11 CLA although this was not significantly different from other groups. The MPO activity proved to be highly variable between animals within the same group.

### Cytokine Production

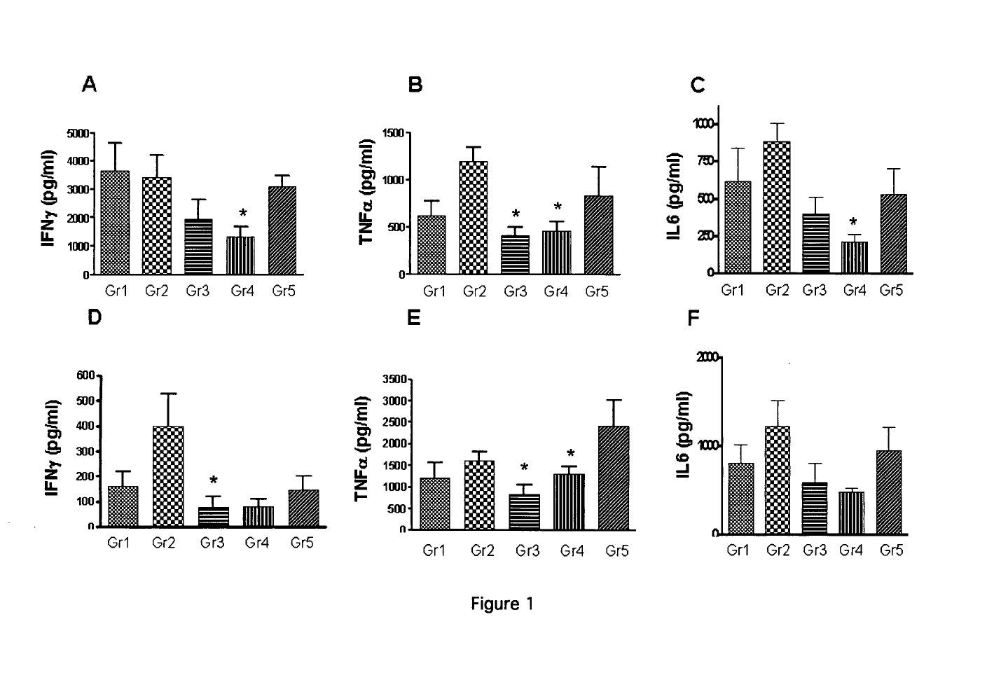

Cytokine analysis was performed on splenocyte supernatants by ELISA following stimulation in vitro with the proinflammatory bacterium S. typhimurium UK1 and with antiCD3-antiCD28 monoclonal antibodies. The proinflammatory cytokines interferon-γ (IFN-γ), tumour necrosis factor α (TNF-α), and interleukin-6 (IL-6) were significantly reduced in the mice fed c9, t11 CLA (group 4) following stimulation with antiCD3-antiCD28 compared to placebo mice (FIG. 1, A-C). IFN-γ levels in the control group were 3093.4 compared with 1334.2 in the c9, t11 CLA group (p<0.01). TNF-α levels in the control group were 838.5 compared with 458.9 in the c9, t11 CLA group (p<0.05). Furthermore, the IL-6 levels in the control group were 528.4 compared with 213.7 in the c9, t11 CLA group (p<0.05). Moreover, TNF-α levels following stimulation with S. typhimurium UK1 showed a significant reduction in the c9, t11 CLA fed group. TNF-α levels in the control group were 2418.8 compared with 1428.7 in the c9, t11 CLA group (p<0.05) (FIG. 1E). IFN-γ and IL-6 were also reduced in this group given c9, t11 CLA following stimulation with S. typhimurium UK1, although these did not reach significant differences (FIG. 1, D and F).

TNF-α levels following stimulation with antiCD3-antiCD28 showed a significant reduction in the probiotic group (group 3). TNF-α levels in the control group were 838.5 compared with 414.1 in the B. breve NCIMB 702258 group (p<0.05) (FIG. 1B). In addition, TNF-α and IFN-γ were significantly reduced in the mice fed B. breve NCIMB 702258 following stimulation with S. typhimurium UK1 (FIG. 1, D-E). TNF-α levels in the control group were 2418.8 compared with 839.0 in the B. breve NCIMB 702258 group (p<0.05). IFN-γ levels in the control group were 145.2 compared with 75.7 in the B. breve NCIMB 702258 group (p<0.05). Moreover, this group given B. breve NCIMB 702258 showed a significant reduction in IL-12 p70 (p<0.05). The mice receiving the probiotic had also a reduction in IL-6, although it did not reach significant difference. There was no reduction in proinflammatory cytokines in the mice fed either LA (group 1) or LA together with B. breve NCIMB 702258 (group 2) compared to control.

### Tissue Fatty Acid Composition

As expected, the mice that were fed pure c9, t11 CLA had a much higher amount of c9, t11 CLA in all tissues (P<0.001). This group had also a decrease in arachidonic acid, 20:4n-6 (AA) in the liver when compared to other groups (Table III) and a significant decrease in 20:4n-6 in cecum digests (P<0.05) (Table IV). The mice within this group had furthermore a significantly higher level of 16:0 (palmitic acid) in the colon and higher levels of 16:1n-7 (palmitoleic acid) in all tissues (data not shown).

The group that was fed LA together with B. breve NCIMB 702258 (group 2), showed an overall higher amount of c9, t11 CLA in all tissues when compared to other groups (excluding the c9, t11 CLA group) (Table V). This group had, similar to the c9, t11 CLA fed group, a significant reduction of 20:4n-6 in cecum digests. Compared to the LA-fed group this group had a lower amount of LA (18:2n-6) in the liver (Table III). Furthermore, compared to the control group this group had a significant decrease in 22:6n-3 (DHA) in cecum digests and colon. The mice given B. breve NCIMB 702258 (group 3) showed a significantly 3-fold higher amount of the beneficial fatty acid 20:5n-3 (EPA) in adipose tissue compared to control (0.45±0.11 compared with 0.15±0.09 g/100 g FAME). They had an approximately 1.5-fold higher amount of EPA in liver, cecum and colon, although these did not reach significant differences. This group had also a significantly higher amount of 22:6n-3 in adipose tissue. In addition, the AA/EPA ratio was lower in the B. breve NCIMB 702258 fed group compared to other groups (Table III and IV). Overall the fatty acid composition was highly variable between animals within the same group.

Effect of the Administration of B. breve NCIMB 702258 on the Amounts of c9t11-CLA in Different Tissues.

When a diet of 1% LA (w/w) was fed, the amounts of c9, t11 CLA in liver, colon and cecum digests harvested 10 weeks after the initiation of B. breve NCIMB 702258 were 4.0-, 3.0- and 2.0-fold higher, respectively, in the mice that received B. breve NCIMB 702258 compared to other groups (Table V). In addition, these mice were the only mice that had c9, t11 CLA incorporated into adipose tissue. They had also a 2-fold increase of c9, t11 CLA in their faeces. Furthermore, in comparison with group 1 that only received LA, this group had a lower amount of LA in their livers (Table III), indicating that LA has been metabolized. Altogether, these results indicate that the LA-conjugation activity in the intestine is due to the administered B. breve NCIMB 702258 and furthermore it confirms the in vivo incorporation of c9, t11 CLA into different tissues.

### Microbial Analysis

Faecal samples were analyzed to assess transit of the probiotic strain. B. breve NCIMB 702258 was recovered in faeces from all mice in group A, within 2 weeks of feeding, confirming survival and transit of the probiotic in the mice. Stool recovery of B. breve NCIMB 702258 was approximately 1×10⁶ CFU/g faeces by week 8 of feeding (FIG. 1). The probiotic strain was not isolated from any of the mice in group B.

### BALB/c Mouse Feeding Trial

### Body Weight

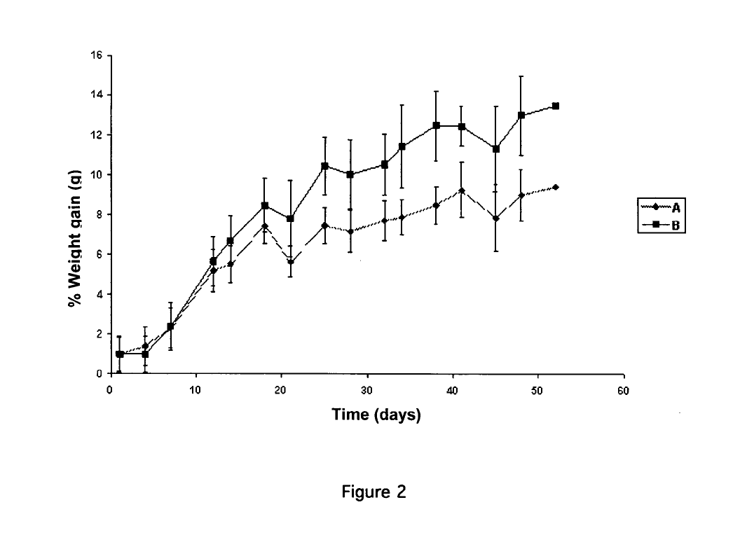

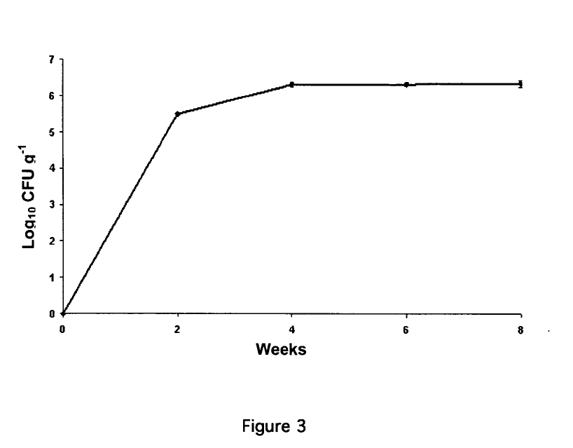

FIG. 2 shows mean body weight development during the experimental period. Body weight increased from 25.3 g to about 27.6 g in group A and from 25.9 g to about 29.4 g in group B. No significant difference was observed between experimental groups.

Effect of the Administration of B. breve NCIMB 702258 on the Amounts of c9t11-CLA in Different Tissues.

The c9, t11 CLA composition of the livers, colons, small intestines and cecum digests are shown in FIG. 3.

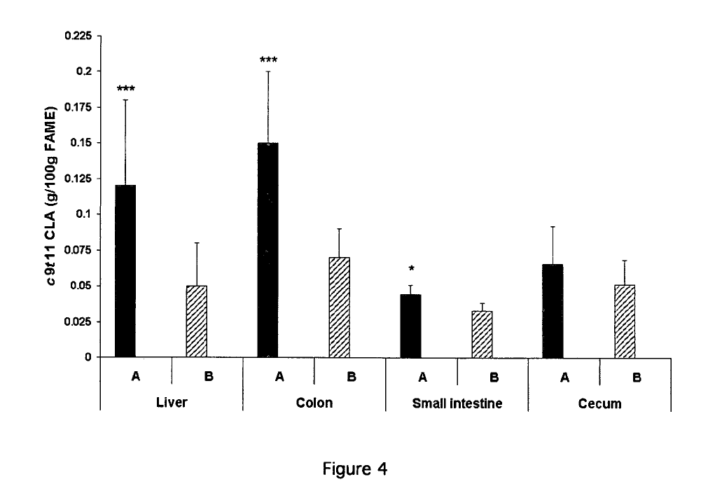

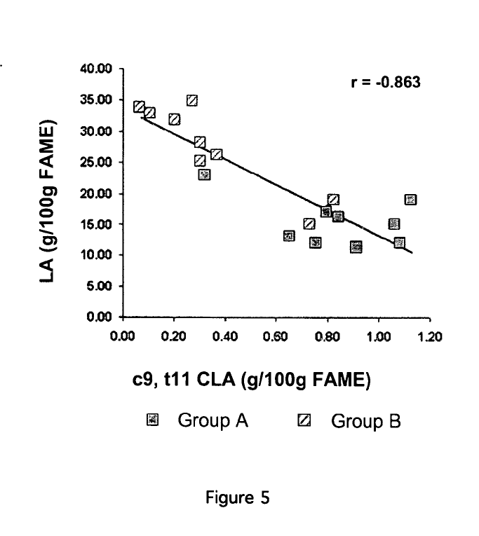

After 8 weeks of feeding there was a significant 2-fold increase of c9, t11 CLA in the livers of the mice that were fed LA together with B. breve NCIMB 702258, compared to placebo that were fed only LA (0.12±0.06 g/100 g FAME compared to 0.05±0.03 g/100 g FAME, p=0.009). This oral administration of B. breve NCIMB 702258 also resulted in a significant 2-fold increase of c9, t11 CLA in the large intestine of group A (0.15±0.05 g/100 g FAME compared to 0.07±0.02 g/100 g FAME, p=0.001). Significantly higher amounts of c9, t11 CLA was also observed in the small intestine of the mice receiving B. breve NCIMB 702258 (0.05±0.01 compared to 0.03±0.01, p=0.04). Furthermore, higher levels of c9, t11 CLA were found in the cecum digesta of the mice within group A compared to group B, although these did not reach significant differences. The mice within group A had also a 2.4-fold significantly higher amount of c9, t11 CLA in their faeces harvested after 8 weeks compared to group B (0.83±0.25 g/100 g FAME compared to 0.35±0.25 g/100 g FAME, p=0.001) (FIG. 4). This higher amount of c9, t11 CLA correlated with a significantly lower amount of LA in the faeces (15.7±3.80 in group A compared to 27.4±7.16 in group B, p=0.001), with the correlation coefficient (r) being −0.863 (FIG. 4). In addition, in comparison with group B that only received LA, group A had a lower amount of LA in the liver, colon, small intestine and cecum digesta, indicating that LA has been metabolized (Table II). c9, t11 CLA was not detected in the heart of any of the mice within group A.

Altogether, these results prove that administration of B. breve NCIMB 702258 to mice increases CLA production in the large intestine, which results in increased CLA absorption and in vivo incorporation of c9, t11 CLA into the liver.

### Tissue Fatty Acid Composition

The fatty acid composition of the livers, colons, small intestines and cecum digestas are presented in Table II.

Apart from an overall higher level of c9, t11 CLA and a lower level of LA in group A compared to group B, there was also a significantly higher level of stearic acid (18:0) in the colon of this group (p=0.02) and an overall lower level of oleic acid (18:1c9) in this group. Furthermore, the mice within this group had a significantly higher amount of eicosapentaenoic acid (EPA, 20:5n-3, p=0.041) and docosahexaenoic acid (DHA, 22:6n-3, p=0.008) in the colon, and also a significantly higher level of dihomo-γ-linolenic acid (20:3n-6, p=0.027). EPA and DHA were also overall higher in the group administered B. breve NCIMB 702258. In addition, the delta-6 desaturation index [(18:3n-6+20:3n-6)/18:2n-6] in the colon was significantly higher in the mice receiving B. breve NCIMB 702258 compared to the placebo group (0.018±0.004 compared to 0.014±0.005, p=0.045).

### Analysis of Other CLA-Producing Strains

Other strains were identified which produce CLA, as follows;

TABLE VI
CLA-producing strains.

| | % conversion to c9, t11 | % |
|---|---|---|
| Strain | CLA | conversion to CALA |
| B. breve DPC 6330 | 76.65 ± 1.75 | ~71 |
| B. breve DPC 6331 | 61.12 ± 3.85 | ~7 |
| B. breve NCIMB | 60% | 45% |
| 702258 | | |
| B. longum DPC 6315 | 60.12 ± 5.14 | none producer |
| B. longum DPC 6320 | 53.08 ± 2.51 | none producer |

### Animal Performance

Mice were weighed twice a week over the 8 week trial period. Oral administration of B. breve NCIMB 702258 and/or α-linolenic acid did not significantly influence body weight gain throughout the trial period.

### Microbial Analysis

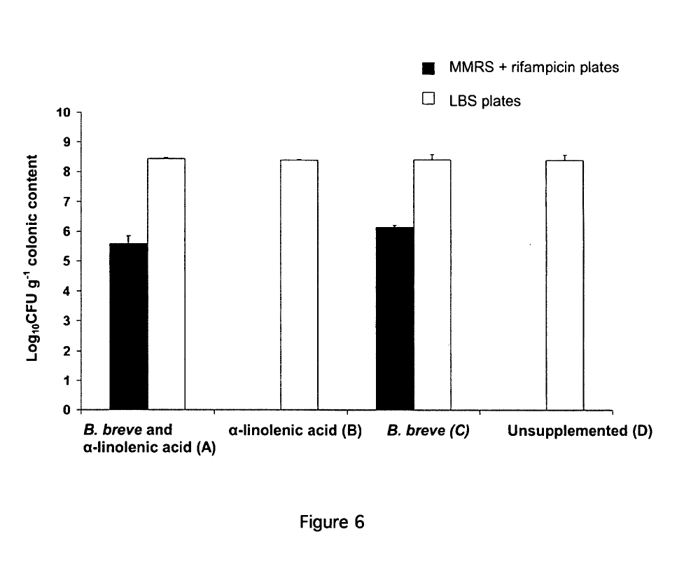

The numbers of B. breve NCIMB 702258 were monitored in the faeces of individual mice every 14 days. The administered B. breve was recovered in faeces from all mice that received the strain, within 2 weeks of feeding, confirming gastrointestinal transit and survival of B. breve NCIMB 702258. Stool recovery of B. breve NCIMB 702258 was approximately 4×10⁵ CFU/g faeces by week 8 of the trial in mice that received B. breve in combination with α-linolenic acid (group A) and approximately 2.2×10⁶ CFU/g faeces in mice that received B. breve without α-linolenic acid (group C) (p>0.05). The B. breve strain was detected in large intestinal contents at ˜4.6×10⁵ CFU/g in mice that received B. breve and α-linolenic acid (group A) and ˜1.4×10⁶ CFU/g in mice that received B. breve alone (group C) at sacrifice (p>0.05) (FIG. 6). B. breve NCIMB 702258 was not isolated from any of the mice within group B (administered α-linolenic acid alone) or group D (placebo control). Following culturing of the large intestinal content on Lactobacillus selective media (LBS), the numbers of CFU obtained did not differ significantly between the groups (p>0.05) (FIG. 6).

### Tissue Fatty Acid Composition

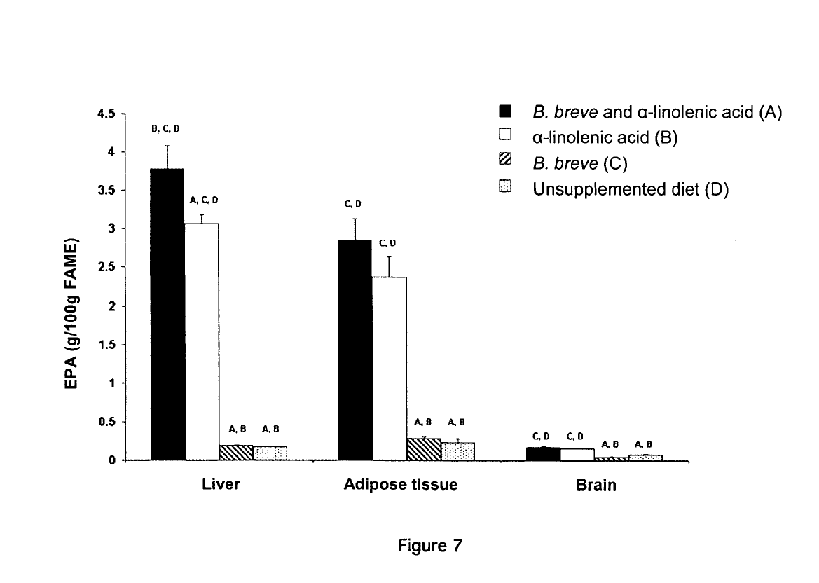

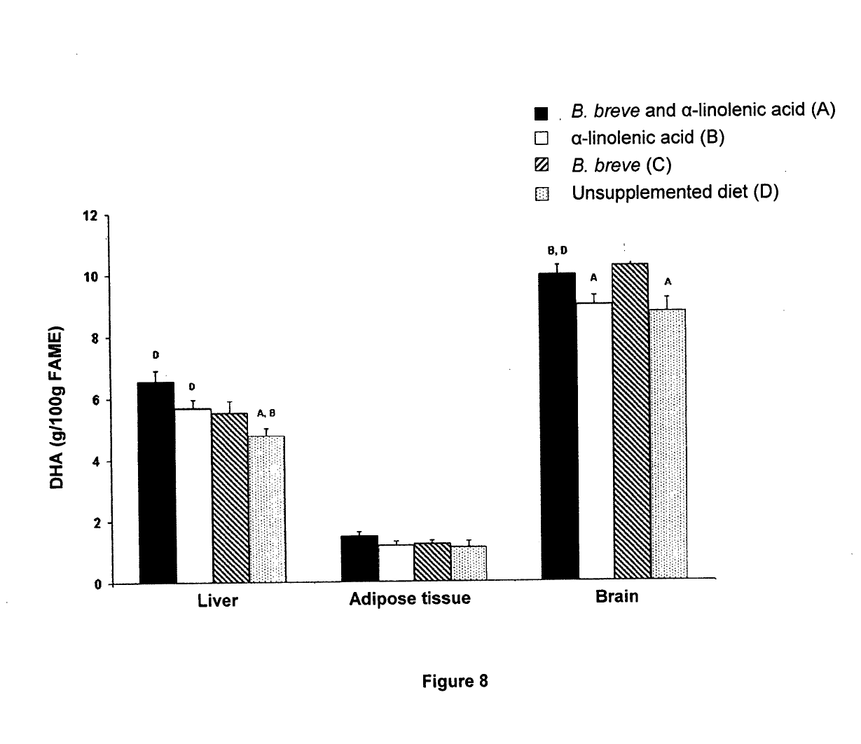

Administration of B. breve in combination with α-linolenic acid resulted in significant changes in the fatty acid composition of host tissues including liver and brain compared to animals administered α-linolenic acid alone. Mice that received B. breve in combination with α-linolenic acid (group A) exhibited 23% higher EPA and 20% higher dihomo-γ-linolenic acid (C20:3n-6) in liver compared with the group administered α-linolenic acid alone (group B, p<0.05, FIG. 7, Table I). The former group also exhibited a 12% higher DHA concentration in brain (p<0.05, FIG. 8) as well as numerically higher concentrations of DHA in adipose tissue and liver (27% and 16%, respectively) (Table II). Furthermore, this group exhibited significantly decreased n-6/n-3 ratio in brain tissue compared with the latter group (p<0.05) (Table II).

Oral administration of B. breve, both in combination with α-linolenic acid and without α-linolenic acid supplementation (group A and C), resulted in significantly higher (p<0.05) concentrations of arachidonic acid (C20:4n-6) and stearic acid (C18:0) incorporated in liver compared with mice that did not receive the bacterial strain (group B and group D) (Table I). Moreover, the mice that received B. breve supplementation, without α-linolenic acid (group C), exhibited numerically higher concentration (16%) of DHA in brain tissue compared with unsupplemented controls (group D), although this was not statistically significant (Table II).

Supplementation of α-linolenic acid, either in combination with B. breve or in the absence of the B. breve strain (group A and group B) resulted in 10-fold higher concentrations of α-linolenic acid and EPA in liver and adipose tissue compared with groups that did not receive the fatty acid supplement (group C and group D) (p<0.001) (Table I). In addition, these groups also exhibited significantly higher concentrations of docosapentaenoic acid (DPA, C22:5n-3) in liver and adipose tissue (p<0.05), significantly higher concentrations of DHA in liver (p<0.05) and significantly lower levels of arachidonic acid in liver, adipose tissue and brain (p<0.05) compared with groups that did not receive α-linolenic acid supplementation (group C and group D) (Table I and II). The arachidonic acid/EPA ratios in liver and adipose tissue were approximately 30-fold and 20-fold lower, respectively, in the groups that received α-linolenic acid (group A and B) compared with animals that did not receive the fatty acid (group D). In addition, the n-6/n-3 ratios were significantly lower in all tissues, except the brain of animals supplemented with α-linolenic acid (group A and B) (p<0.001). Supplementation with α-linolenic acid also resulted in significant decreases in concentrations of palmitic acid (C16:0), palmitoleic acid (C16:1c9) and oleic acid (C18:1c9) (p<0.001), and significantly higher stearic acid (C18:0) in liver compared with unsupplemented groups (p<0.001) (Table I).

### Discussion

This study sought to investigate whether administration of a CLA-producing strain would produce CLA in vivo and furthermore whether this bacterial produced CLA would ameliorate colonic inflammation. Although, only limited data on the effects of probiotics on dietary fatty acids has been published, there are indications that intestinal bacteria may interact with different fatty acids. To our knowledge, this is a first-time observation where the effects of oral intake of CLA-producing bacteria on inflammatory bowel disease have been investigated.

Certain metabolic capabilities of microorganisms that easily are observed in vivo do not necessarily occur in vitro. However, results from this study prove that linoleic acid has been converted to c9t11-CLA by B. breve NCIMB 702258 in vivo and furthermore it confirms the in vivo incorporation of c9t11-CLA into different tissues. Administration of B. breve NCIMB 702258 to mice increased the CLA production in the large intestine threefold and the CLA content of the liver fourfold. Altogether, this proves that administration of B. breve NCIMB 702258 to mice increases CLA production in the large intestine which results in increased CLA absorption. This is in agreement with a study made by Fukuda et al. (30), who administered a CLA-producing strain, Butyrivibrio fibrisolvens MDT-5, every other day to mice for two weeks. This oral administration resulted in increased amounts of CLA in the contents of the large intestine (2.5-fold) as well as in adipose tissue (threefold). Feeding a high-LA diet, as well as prolonging the period of MDT-5 administration, further increased the CLA content in body fat. The MDT-5 strain is a bovine bacterium and not a human strain, like those of the invention, so it is not part of the human gut microflora. Fukuda et al did not show production of CLA in the liver or any effects on inflammation. A subsequent study in rats concluded that gut microbes did not lead to increased CLA production in vivo. In contrast, a study made by Kamlage et al. (31) concluded that intestinal microorganisms do not supply rats with systemic CLA, insofar as CLA did not accumulate in tissues upon administration of LA conjugating microbes to germ-free rats. They found however that LA-conjugation activity in feces was increased after administrating microbes known to produce CLA. They concluded that this may be due to the inability of the microorganism investigated to conjugate LA in vivo. However, the strain used in this study, B. breve NCIMB 702258, proved to conjugate LA into CLA in SCID mice in vivo. Little is known about the intestinal absorption of CLA. However, the intestines are the first step of nutrient delivery to tissues and, as such, may modulate the bioavailability of ingested fatty acids and also, therefore, their biological effects. The absorption of c9, t11 CLA and t10, c12 CLA in the small intestine is particularly unclear (49). Since LA has proinflammatory capabilities and has furthermore been reported to act as a promoter of carcinogenesis, conversion of LA to CLA by bacteria in the intestine may be important to reduce LA absorption following ingestion of high-LA acid diets. In the typical western diet, 20-25 fold more n-6 fats than n-3 fats are consumed. This predominance of n-6 fat is due to the abundance in the diet of LA, which is present in high concentrations in soy, corn, safflower, and sunflower oils. LA is converted to arachidonic acid (AA) and AA is the precursor for the proinflammatory eicosanoids prostaglandin E₂ (PGE₂) and leukotriene B₄ (LTB₄), which are maintained at high cellular concentrations by the high n-6 and low n-3 polyunsaturated fatty acid content of the modern western diet. Furthermore, in order to maintain health, CLA needs to be taken continually, but large doses of CLA as a supplement may have deleterious effect. Therefore, it is desirable to absorb CLA produced slowly and continually in the large intestine. This study shows that B. breve NCIMB 702258 may be useful as a probiotic to provide CLA in the large intestine continuously.

Although CLA was produced in the group fed B. breve NCIMB 702258 and LA, these mice did not show any amelioration of the proinflammatory cytokines IL-6, IFN-γ and TNF-α. This is probably due to the high amount of LA fed. As mentioned above, LA has been shown to have proatherogenic and proinflammatory properties. For example LA has been shown to increase the activation of NF-κB and expression of cytokines and cell adhesion molecules.

Consistent with previous observations in liver of mice (35), the analysis of the fatty acid composition of liver in this study revealed that the dietary CLA supplementation to one group of mice decreased the concentration of arachidonic acid (AA). The mice fed c9, t11 CLA had a significantly lower amount of AA in the cecum and furthermore a lower amount incorporated in their livers. AA is a precursor for the generation of first-phase eicosanoids (i.e., two series prostaglandins and four series leukotrienes) involved in early microinflammatory events (i.e., polymorpho-nuclear neutrophilic leukocyte chemotaxis and release of superoxide anions). Enhanced intestinal eicosanoid concentrations closely correlate with severe signs of colonic inflammation. Results from this study show that dietary intake of c9, t11 CLA inhibits hepatic and intestinal 20:4(n-6) synthesis. This may in turn down-regulate the production of pro-inflammatory eicosanoids. The beneficial impact of c9, t11 CLA on the mucosa was associated with changes in the systemic cytokine production in vitro. Following challenge (either with antiCD3-antiCD28 monoclonal antibodies or S. typhimurium UK1), there was a reduction in the Th1 cytokines TNF-α and IFN-γ and in the Th2 cytokine IL-6 in the mice fed this fatty acid. Research in several species, including poultry (38), rats (39), and mice (40), have shown that dietary CLA can reduce the release of proinflammatory cytokines. At the molecular level, the concentration of cytokines in tissues is controlled in part by mechanism(s) of transcriptional regulation. CLA works as an activator for PPAR-γ. PPAR-γ activation has been demonstrated to antagonize the activities of several transcription factors including NF-κB. As a result of this interference with the NF-κB signalling pathway, the expression of proinflammatory cytokines (i.e., TNF-α, IL-6 and IL-1β is suppressed and macrophage apoptosis induced, both effects with likely consequences in inflammation.

In addition, the activity of MPO (an index of tissue-associated neutrophil accumulation) was also lower in the mice fed c9, t11 CLA.

Although we did not notice any alleviation of the proinflammatory cytokines in the mice fed B. breve NCIMB 702258 and LA (probably due to the high amount of LA fed), we noticed a positive effect on the mucosal inflammation in the group fed only B. breve NCIMB 702258. This probiotic effect was reflected by a reduction in the proinflammatory cytokines IL-6, IFN-γ, IL-12p70 and TNF-α secreted by splenocytes. Alteration in cytokine profiles observed is important as IBD is associated with a predominance of Th1 cytokines (e.g. IFN-γ, IL-12p70 and TNF-α). Previous probiotic trials have shown the same probiotic efficacy in inflammatory disorders as seen in this study. For example, in a study by McCarthy et al. (29) both Lactobacillus salivarius 433118 and Bifidobacterium infantis 35624 attenuated colitis through a significant amelioration of proinflammatory IFN-γ and TNF-α. These strains however did not produce CLA. Treatment of IL-10 knockout mice with VSL#3, Lactobacillus plantarum 299v and Escherichia coli ssp. laves have demonstrated improvements in inflammation and histological disease in conjunction with significantly decreased mucosal secretions of IFN-γ and TNF-α(45-47). The MPO-activity was also lower in the mice fed B. breve NCIMB 702258 signifying the probiotic-mediated immune modulation by this strain.

Interestingly, we noticed an altered PUFA composition in the group given B. breve NCIMB 702258 compared to the control group. This alteration included a threefold significantly higher amount of the beneficial fatty acid 20:5n-3 (EPA) in the adipose tissue. The mice within this group had also a higher amount of EPA in liver, cecum and colon, and a significantly higher amount of DHA in adipose tissue. In similar action to CLA, 20:5n-3 also works as an activator of PPARs. For example, EPA-activated PPAR-γ induces lipoprotein lipase and fatty acid transporters and enhances adipocyte differentiation as well as inhibits the function of the transcription factor NF-κB and cytokines. Furthermore, the AA/EPA ratio was much lower in the B. breve NCIMB 702258 fed group compared to other groups. This suggests that B. breve NCIMB 702258 modifies the fatty acid composition to a more beneficial composition. Although only limited data on the effects of probiotics on dietary fatty acids has been published, there are indications that intestinal bacteria may interact with different fatty acids. There are at least two possible mechanisms by which B. breve NCIMB 702258 could achieve the effects seen in the present study; (i) B. breve NCIMB 702258 could influence the fatty acid composition by either utilizing or simply assimilating certain PUFAs, or (ii) B. breve NCIMB 702258 could influence the mechanisms of dietary PUFA uptake to the intestinal epithelium. Whether this modulation in fatty acid composition by B. breve NCIMB 702258 is the responsible mechanism for the probiotic attenuation of colitis seen here requires further validation.

Proinflammatory cytokine production by splenocytes was significantly reduced in the groups fed the probiotic strain B. breve NCIMB 702258 and pure cis-9, trans-11 CLA. Consumption of B. breve NCIMB 702258 resulted in a significant amelioration of the proinflammatory Th1 cytokine tumour necrosis factor cc (TNF-α) and a reduction of the proinflammatory Th1 cytokine interferon-γ (IFN-γ) compared to placebo. Consumption of c9, t11 CLA lead to a significant reduction in IFN-γ, TNF-α, and interleukin-6 (IL-6).

The group given B. breve NCIMB 702258 had a significantly threefold higher amount of the beneficial fatty acid 20:5n-3 (EPA) in adipose tissue compared to placebo. In addition, the arachidonic acid (AA)/eicosapentaenoic acid (EPA) ratio was lower in this group compared to other groups.

In the group fed B. breve NCIMB 702258 together with LA, there was a significant increase of c9t11-CLA in the liver compared to other groups (excluding the c9, t11 fed group). This group had also an increase of c9t11-CLA in colon (threefold) and was furthermore the only group that had c9t11-CLA incorporated into the adipose tissue. Altogether these results prove that LA has been converted to c9t11-CLA by B. breve NCIMB 702258 in vivo. This group didn't show any amelioration in the immune response however which could be due to the high amount of LA fed.

Linoleic acid (LA; cis-9, cis-12-18:2) is metabolised by bacteria in the rumen of ruminants in a process known as biohydrogenation. This process carries important implications for the fatty acid composition of milk. The first step in the biohydrogenation by the rumen bacteria is the isomerisation of the cis-12 double bond of LA to a trans-11 configuration resulting in c9, t11 CLA (cis-9, trans-11-18:2). Next step is a reduction of the cis-9 double bond resulting in a trans-11 fatty acid; vaccenic acid (VA; trans-11-18:1). Both this fatty acids are considered to be beneficial for health. The final step in the biohydrogenation is a further hydrogenation of the trans-11 double bond in VA, producing stearic acid (18:0) as a final product. The microbiology of biohydrogenation in the rumen has received lots of attention, but similar research has not been carried out for the human intestinal microflora. CLA has been shown to exert a variety of beneficial biological activities in several experimental animal models. In this study we investigated whether a c9, t11-CLA producing Bifidobacterium strain of human origin; B. breve NCIMB 702258 could produce the bioactive c9, t11-CLA from LA in vivo.

This study shows that an eight week oral treatment with the CLA-producing strain B. breve NCIMB 702258 increases the content of c9, t11 CLA in the colon, small intestine, and liver significantly, proving the in vivo c9, t11-CLA production by this strain. This is in agreement with a study made by Fukuda et al. (30). They reported that CLA was produced from LA when human faeces were incubated in vitro, but substantially no CLA was produced when the same fecal bacteria were introduced into germ-free rats. They stated that the difference between the in vivo and in vitro results is explainable by the fact that CLA production is inhibited by glucose (16).

It is now well established that CLA has antiproliferative and anti-inflammatory effects on colonocytes, so the provision of CLA in the intestinal lumen would be considered beneficial, particularly for inflammatory bowel disease, such as ulcerative colitis and Crohn's disease. B. breve NCIMB 702258 is expected to continuously produce CLA after it colonises the gut, thereby continuously exerting beneficial effects from CLA. Furthermore, in order to maintain health, CLA needs to be taken continually, but large doses of CLA as a supplement may have deleterious effect. Therefore, it is desirable to supply a low level of CLA frequently or continuously to the body. This study shows that B. breve NCIMB 702258 may be useful as a probiotic to provide CLA in the large intestine continuously.

In addition, since LA has proinflammatory capabilities and have furthermore been reported to act as a promoter of carcinogenesis, conversion of LA to CLA by bacteria in the intestine may be important to reduce LA absorption following ingestion of high-LA acid diets. The mice receiving B. breve NCIMB 702258 had an overall lower amount of LA in the liver, colon, small intestine and cecum digesta compared to the LA-fed group, indicating that LA has been metabolized by B. breve NCIMB 702258.

The amount of LA available for CLA production in the colon is varying and is dependant upon the amount ingested and the efficacy of absorption in the small intestine. Edionwe et al. (22) showed that humans generally excrete ˜20 mg of LA/day, suggesting that substrate is available for microbial production of CLA.

Stearic acid, the final product of LA metabolism by bacteria, was significantly higher in the colon of the mice fed B. breve NCIMB 702258. This is in agreement with a study made by Howard et al. (21). They showed that bacteria from the human colon can hydrogenate LA to stearic acid. In their study, human faecal suspensions were incubated with LA for 4 h. As a result of this incubation, LA was significantly decreased and there was a significant rise in its hydrogenation product, stearic acid.

We also noticed an altered PUFA composition in the mice given B. breve NCIMB 702258 compared to the control group. This alteration included for example significantly higher levels of the beneficial fatty acids eicosapentaenoic acid (EPA) and docosahexaenoic acid (DHA) in the colon. In similar action to CLA, EPA also works as an activator of PPARs. For example, EPA-activated PPAR-γ induces lipoprotein lipase and fatty acid transporters and enhances adipocyte differentiation as well as inhibits the function of the transcription factor NF-κB and cytokines The delta-6 desaturation index [(18:3n-6+20:3n-6)/18:2n-6] in the colon was also higher in the mice receiving the Bifidobacterium strain. Fukushima et al. concluded that feeding a probiotic mixture of organisms (Bacillus, Lactobacillus, Streptococcus, Clostridium, Saccharomyces, and Candida) increases the delta-6 desaturase activity in the liver of rats (30).

The influence of dietary PUFA on phospholipids, their eicosanoid derivatives and the transmembrane-signalling lipid rafts into which they are arranged provide multiple mechanisms for dietary modulation of the balance of inflammatory mediators in the human gut. In addition, gut mucosal inflammation is now recognized as being heavily influenced by the composition of the gastrointestinal microbiota (21, 22). Although probiotics and dietary PUFA have been considered separately for the management of inflammation, we propose that these two interventions may act synergistically. To investigate this, we administered B. breve NCIMB 702258 in combination with α-linolenic acid to mice in order to assess how this combination would affect the EPA and DHA composition of different host tissues. We found that dietary supplementation with B. breve NCIMB 702258 in combination with α-linolenic acid resulted in modulation of host fatty acid composition, and particularly resulted in significantly higher EPA and dihomo-γ-linolenic acid in liver and higher DHA in brain compared to mice that received α-linolenic acid without microbial supplementation. Kaplas et al. (8) demonstrated that administration of probiotics (L. rhamnosus GG and B. animalis subsp. lactis Bb12) to pregnant women resulted in higher concentrations of dihomo-γ-linolenic acid in placental fatty acids. Interestingly, the n-6/n-3 ratio in brain was also significantly lower in mice that received B. breve in combination with α-linolenic acid compared to mice supplemented with α-linolenic acid alone. Moreover, oral administration of B. breve, both in combination with α-linolenic acid and without α-linolenic acid supplementation, resulted in significantly higher amounts of arachidonic acid incorporated in liver compared to mice that did not receive B. breve. Given that administration of B. breve resulted in significantly higher concentrations of long-chain PUFA such as EPA, DHA and also arachidonic acid, administration of this strain resulted in an increase in the levels of unsaturation within fatty acids. Interestingly, it was previously shown that a variety of probiotics increased the activity of liver Δ6-desaturase in rats, which resulted in increased amounts of arachidonic acid derived from linoleic acid (23).

Supplementation of α-linolenic acid both in combination with B. breve and in the absence of the B. breve strain resulted in significant increases in EPA and DHA in the liver and adipose tissues, at the expense of arachidonic acid. Since EPA replaces arachidonic acid as an eicosanoid substrate in cell membranes of platelets, erythrocytes, neutrophils, monocytes and hepatocytes (24), this results in a reduced synthesis of inflammatory eicosanoids from arachidonic acid and subsequently elevated production of anti-inflammatory eicosanoids from EPA. This alteration towards a more anti-inflammatory profile could be of importance in a variety of chronic inflammatory settings such as inflammatory bowel disease, rheumatoid arthritis, multiple sclerosis, Alzheimer's disease and certain psychiatric diseases such as depression, which are characterized by an excessive production of arachidonic acid-derived eicosanoids (25-27). Moreover, since excessive intake of n-6 PUFA, characteristic of modern Western diets, could potentiate inflammatory processes and so could predispose to or exacerbate associated diseases, increasing the intake of α-linolenic acid and/or EPA may have a protective effect. Supplementation with α-linolenic acid was also associated with a decrease in palmitic acid, palmitoleic acid and oleic acid in liver and adipose tissue, and higher concentrations of stearic acid in these tissues. Similar findings were obtained by Fu and Sinclair (28) who reported that guinea pigs fed a high α-linolenic acid diet had significantly lower levels of palmitic acid in liver and adipose tissue compared to guinea pigs fed a low α-linolenic acid diet.

Since the effect of combined B. breve and α-linolenic acid intervention on EPA- and DHA concentrations was greater than that of α-linolenic acid intervention alone, this effect could be attributed to B. breve NCIMB 702258 and thus suggest that feeding a metabolically active strain can influence the fatty acid composition of host tissues. The mechanism by which B. breve NCIMB 702258 mediated the changes in host n-3 fatty acid composition seen in the present study remains unclear. A possible mechanism may be the properties of bacteria in regulating desaturase activity involved in the metabolism of fatty acids (23).

In conclusion, the present study shows that administration of B. breve NCIMB 702258 is associated with alterations in the fatty acid composition of host liver and brain. This study suggests a definite role for the interactions between PUFAs and commensal bacteria. This “synergistic” effect of commensals and fatty acids could be of therapeutic beneficial for a range of immuno-inflammatory disorders as well as having significance for the promotion of neurological development in infants

### CONCLUSION

The presented study opens possibilities to improve the quality of life of IBD patients by using probiotic bacteria. The results suggest that some physiological effects of probiotics e.g. immunomodulating properties, may be associated with physiological interactions between probiotics and polyunsaturated fatty acids (PUFA). Furthermore, this study provides evidence for the in vivo CLA production by B. breve NCIMB 702258

Thus the introduction of a CLA-producing probiotic strain may enhance the CLA production in the large intestine and could possible contribute to the anti-inflammatory effect and furthermore the prevention of inflammatory bowel disease. The results from this study suggest a definite role for interactions between fatty acids and commensals. Furthermore, that data indicate that the synergist effect between administered microbes and fatty acids could result in more efficient probiotic preparations, and indicate the anti-inflammatory potential of this dietary combination.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

TABLE I
Dietary treatments of different groups (n = 8).

| | |
|---|---|
| Group 1 | 1% LA in standard diet |
| Group 2 | 1% LA in standard diet and B. breve |
| | NCIMB 702258 (a daily dose of 10⁹ |
| | organisms) |
| Group 3 | Standard diet and B. breve NCIMB |
| | 702258 (a daily dose of 10⁹ organisms) |
| Group 4 | 1% c9, t11 CLA in standard diet |
| Group 5 | Standard diet |

TABLE II
Colonic mucosal Myeloperoxidase activity (MPO, U/mg protein).

| | MPO colon |
|---|---|
| Group | (U/mg protein) |
| 1 (LA) | 1.16 ± 0.23 |
| 2 (LA + probiotic) | 0.98 ± 0.57 |
| 3 (probiotic) | 0.95 ± 0.86 |
| 4 (c9, t11 CLA) | 0.86 ± 0.73 |
| 5 (placebo) | 2.18 ± 1.51 |

Data are expressed as mean ± SD (n = 8).

TABLE III
Bioproduction of c9, t11 CLA by B. breve NCIMB 702258 occurs
in vivo.

| Tissue | 1 (LA) | 2 (LA + 702258) | 3 (702258) | 5 (Placebo) |
|---|---|---|---|---|
| Liver | 0.01 ± 0.01² | 0.04 ± 0.03^{1,3,5} | 0.01 ± 0.01² | ND² |
| White | ND | 0.01 ± 0.02 | ND | ND |
| Adipose | | | | |
| Colon | ND | 0.03 ± 0.04 | ND | ND |
| Cecum | 0.08 ± 0.02 | 0.16 ± 0.23 | 0.06 ± 0.04 | 0.07 ± 0.06 |
| digesta | | | | |
| Faeces | 0.09 | 0.15 | 0.04 | 0.09 |

Data are expressed as mean ± SD (n = 8), g/100 g FAME.
^{1,2,3,5} Different superscript numbers within a column indicate significant difference (n = 8, P <0.05).
ND = not detected.

TABLE IV
Fatty acid composition (%) of liver and adipose tissue from BALB/c mice

| | Liver | | | |
|---|---|---|---|---|
| FAME | A | B | C | D |
| C16:0 | 23.11 ± 0.99^{c,d} | 23.89 ± 0.71^{c,d} | 27.78 ± 0.64^{a,b} | 27.64 ± 0.42^{a,b} |
| C16:1c9 | 1.56 ± 0.10^{b,c,d} | 2.07 ± 0.18^{a,d} | 2.32 ± 0.18^{a} | 2.73 ± 0.27^{a,b} |
| C18:0 | 13.83 ± 0.42^{b,c,d} | 12.36 ± 0.44^{a,d} | 11.50 ± 0.35^{a,d} | 10.40 ± 0.35^{a,b,c} |
| C18:1c9 | 9.16 ± 0.36^{c,d} | 10.16 ± 0.62^{c,d} | 13.75 ± 0.57^{a,b} | 15.16 ± 0.75^{a,b} |
| C18:2n-6 | 18.70 ± 0.35 | 18.32 ± 0.21^{d} | 18.21 ± 0.32^{d} | 19.38 ± 0.42^{b,c} |
| C18:3n-3 | 8.37 ± 0.91^{c,d} | 9.47 ± 0.64^{c,d} | 0.50 ± 0.03^{a,b} | 0.58 ± 0.04^{a,b} |
| C18:3n-6 | 0.20 ± 0.02^{c,d} | 0.17 ± 0.01^{c,d} | 0.30 ± 0.03^{a,b} | 0.32 ± 0.01^{a,b} |
| C18:4n-3 | 0.14 ± 0.01^{c,d} | 0.16 ± 0.01^{c,d} | 0.23 ± 0.03^{a,b} | 0.22 ± 0.02^{a,b} |
| C20:3n-6 | 0.73 ± 0.05^{b} | 0.61 ± 0.02^{a} | 0.69 ± 0.04 | 0.63 ± 0.03 |
| C20:4n-6 | 6.71 ± 0.29^{b,c,d} | 5.57 ± 0.14^{a,c,d} | 11.45 ± 0.58^{a,c,d} | 9.78 ± 0.53^{a,b,c} |
| C20:5n-3 | 3.77 ± 0.30^{b,c,d} | 3.07 ± 0.12^{a,c,d} | 0.19 ± 0.01^{a,b} | 0.17 ± 0.02^{a,b} |
| C22:5n-3 | 1.02 ± 0.06^{c,d} | 0.94 ± 0.05^{c,d} | 0.23 ± 0.02^{a,b} | 0.26 ± 0.02^{a,b} |
| C22:6n-3 | 6.56 ± 0.35^{d} | 5.66 ± 0.28^{d} | 5.51 ± 0.41 | 4.76 ± 0.24^{a,b} |
| n-6/n-3 | 1.36 ± 0.10^{c,d} | 1.30 ± 0.07^{c,d} | 4.70 ± 0.23^{a,b} | 5.07 ± 0.16^{a,b} |

| | Adipose tissue | | | |
|---|---|---|---|---|
| FAME | A | B | C | D |
| C16:0 | 28.84 ± 2.83^{c} | 28.73 ± 2.16^{c} | 30.92 ± 0.82^{a,b,d} | 29.87 ± 0.83^{c} |
| C16:1c9 | 5.39 ± 0.73 | 6.07 ± 2.01 | 6.69 ± 1.95 | 6.54 ± 2.31 |
| C18:0 | 8.76 ± 1.00 | 8.00 ± 2.33 | 7.71 ± 1.99 | 7.31 ± 2.87 |
| C18:1c9 | 14.41 ± 1.29 | 14.83 ± 3.01 | 16.92 ± 3.07 | 18.35 ± 4.84 |
| C18:2n-6 | 18.22 ± 0.25^{c} | 18.80 ± 0.81^{c} | 16.56 ± 0.77^{a,b} | 17.96 ± 3.39 |
| C18:3n-3 | 6.56 ± 1.00^{c,d} | 7.35 ± 2.15^{c,d} | 0.66 ± 0.10^{a,b} | 0.79 ± 0.28^{a,b} |
| C18:3n-6 | 0.15 ± 0.02 | 0.13 ± 0.03 | 0.15 ± 0.03 | 0.14 ± 0.04 |
| C18:4n-3 | 0.16 ± 0.02 | 0.12 ± 0.03 | 0.14 ± 0.04 | 0.14 ± 0.03 |
| C20:3n-6 | 0.37 ± 0.04 | 0.30 ± 0.09 | 0.32 ± 0.09 | 0.29 ± 0.16 |
| C20:4n-6 | 6.20 ± 0.97^{c} | 5.27 ± 2.04^{c,d} | 10.57 ± 3.45^{a,b} | 9.29 ± 5.27^{b} |
| C20:5n-3 | 2.86 ± 0.27^{c,d} | 2.37 ± 0.76^{c,d} | 0.29 ± 0.07^{a,b} | 0.23 ± 0.15^{a,b} |
| C22:5n-3 | 0.67 ± 0.06^{c,d} | 0.55 ± 0.14^{c,d} | 0.27 ± 0.03^{a,b} | 0.27 ± 0.16^{a,b} |
| C22:6n-3 | 1.49 ± 0.12 | 1.17 ± 0.36 | 1.22 ± 0.30 | 1.10 ± 0.59 |
| n-6/n-3 | 2.20 ± 0.21^{c,d} | 2.15 ± 0.15^{c,d} | 10.80 ± 0.28^{a,b} | 11.56 ± 0.92^{a,b} |

Results are expressed as means (SEM) g/100 g FAME (n = 8).
^{a,b,c,d}Different superscript numbers within a column indicate significant difference (n = 8, p < 0.05). FAME = fatty acid methyl esters. Group A = 1% α-linolenic acid in combination with 1 × 10⁹ live B. breve NCIMB 702258 per day. group B = 1% α-linolenic acid, Group C = standard diet in combination with 1 × 10⁹ live B. breve NCIMB 702258, and group D = unsupplemented mice. C16:0 palmitic acid; C16:1c9 palmitoleic acid; C18:0 stearic acid; C18:1c9 oleic acid; C18:2n-6 linoleic acid; C18:3n-3 linolenic acid; C18:3n-6γ-linolenic acid; C18:4n-3 stearidonic acid; C20:3n-6 dihomo-γ-linolenic acid; C20:4n-6 arachidonic acid; C20:5n-3 eicosapentaenoic acid; C22:5n-3 docosapentaenoic acid; C22:6n-3 docosahexaenoic acid.

TABLE V
Fatty acid composition (%) of brain from BALB/c mice

| | Brain | | | |
|---|---|---|---|---|
| FAME | A | B | C | D |
| C16:0 | 30.69 ± 0.65 | 32.38 ± 0.44^{c} | 28.88 ± 0.90^{b,d} | 31.47 ± 0.51^{c} |
| C16:1c9 | 0.81 ± 0.02^{a,b} | 0.94 ± 0.03^{a,c,d} | 0.73 ± 0.03^{a,d} | 0.84 ± 0.03^{b,c} |
| C18:0 | 20.22 ± 0.07^{b} | 19.87 ± 0.14^{a} | 20.11 ± 0.20 | 19.85 ± 0.28 |
| C18:1c9 | 17.58 ± 0.19 | 17.61 ± 0.24 | 17.14 ± 0.29 | 17.23 ± 0.33 |
| C18:2n-6 | 1.18 ± 0.04^{b,c} | 1.54 ± 0.10^{a,c,d} | 1.00 ± 0.05^{a,b} | 1.14 ± 0.03^{b} |
| C18:3n-3 | 0.10 ± 0.01^{b,c,d} | 0.18 ± 0.02^{a,c,d} | ND | ND |
| C18:3n-6 | ND | ND | ND | ND |
| C18:4n-3 | ND | ND | ND | ND |
| C20:3n-6 | ND | ND | ND | ND |
| C20:4n-6 | 7.06 ± 0.07^{c} | 6.88 ± 0.12^{c,d} | 7.84 ± 0.23^{a,b} | 7.37 ± 0.17^{b} |
| C20:5n-3 | 0.17 ± 0.02^{c,d} | 0.15 ± 0.01^{c,d} | 0.04 ± 0.01^{a,b} | 0.07 ± 0.01^{a,b} |
| C22:5n-3 | 0.25 ± 0.01^{c,d} | 0.24 ± 0.01^{c} | 0.11 ± 0.01^{a,b} | 0.17 ± 0.04^{a} |
| C22:6n-3 | 9.95 ± 0.30^{b,d} | 8.87 ± 0.30^{a} | 10.15 ± 0.75 | 8.74 ± 0.43^{a} |
| n-6/n-3 | 0.79 ± 0.03^{b,d} | 0.89 ± 0.03^{a} | 0.87 ± 0.04 | 0.97 ± 0.04^{a} |

Results are expressed as means (SEM) g/100 g FAME (n = 8).
^{a,b,c,d}Different superscript numbers within a column indicate significant difference (n = 8, p < 0.05).
Group A = 1% α-linolenic acid in combination with 1 × 10⁹ live B. breve NCIMB 702258 per day.
group B = 1% α-linolenic acid, Group C = standard diet in combination with 1 × 10⁹ live B. breve NCIMB 702258, and group D = unsupplemented diet.
ND = not detected.

### REFERENCES

1. Rembacken et. al. 1999. Lancet, 354: 635-9.
2. Malchow H A.
3. Giochetti, P., Rizzello, F., Venturi, A. et al. 2000. Gastroenterology, 119:305-309.
4. Hontecillas, R., Zimmermann, D. R., Hutto, D. L., Wilson, J., Ahn, D. U., Bassaganya-Riera, J. 2002. J. Nutr. 132: 2019-2027.
5. Kew, S., Gibbons, E. S., Thies, F., McNeill, G. P., Quinlan, P. T. and Calder, P. C. 2003. Br J Nutr, 90:1071-80.
6. Bassaganya-Riera, J., Hontecillas, R. and beitz, D. C. 2002. Clin. Nutr. 21, 451-459.
7. Kamlage, B., Hartmann, L., Gruhl, B., Blaut, M. 2000. J. Nutr. 130: 2036-2039.
8. Bassaganya-Riera et al. 2004. Gastroenterology, 127:777-791.
9. Krawisz, J., Sharon, P. and Stenson, M. 1984. Gastroenterology. 87: 1344-1350.
10. Folch, J., Lees, M., and Sloane Stanley, G. H. 1957. J. Biol. Chem. 226, 497-509.
11. Park, P. W. and Goins, R. E. 1994. J. Food Sci. 59 (6): 1262-1266.
12. Kruskal, W. H. and Wallis, W. A. 1952. J. Am. Stat. Assoc. 47: 583-621.
13. McCarthy et. al. 2003. Gut. 52:975-980.
14. Fukuda, S., Suzuki, Y., Murai, M., Asanuma, N. and Hino, T. 2006. J. Appl. Micro. 100, 787-794.
15. Kamlage, B., Hartmann, L., Gruhl, B. and Blaut, M. 1999. J. Nutr. 129: 2212-2217.
16. Belury, M. A. and Kempa-Steczko, A. 1997. Lipids, 32: 199-204.
17. Takahashi, K., Kawamata, K., Akiba, Y., Iwata, T. & Kasai, M. 2002. Br. Poult. Sci. 43:47-53.
18. Turek, J. J., Li, Y., Schoenlein, I. A., Allen, K. G. D. & Watkins, B. A. 1998. J. Nutr. Biochem. 9:258-266.
19. Akahoshi, A., Goto, Y., Murao, K., Miyazaki, T., Yamasaki, M., Nonaka, M., Yamada, K. & Sugano, M. 2002. Biosci. Biotechnol. Biochem. 66:916-920.
20. McCarthy et. al. 2003. Gut. 52:975-980.
21. Madsen K, Cornish A, Soper P, et al. 2001. Gastroenterology. 121:580-91.
22. Schultz M, Veltkamp C, Dieleman L A, W B, et al. 2002 Inflamm Bowel Dis 8:71-80.
23. Konrad A, Mahler M, Flogerzi B, et al. 2003. Scand J. Gastroenterol. 38:172-9.
24. Bradford, M. 1976. Anal Biochem. 72:248-254.
25. Tsuzuki, T. and Ikeda, I. 2007. Biosci. Biotech. Biochem. 71:70238-1-7.
26. Rada, V. 1997. Biotechnol Tech 11:909-912.
27. Chin, S. F., Storkson, J. M., Liu, W., Albright, K. J., Pariza, M. W. 1994. J. Nutr. 124:694-701.
28. Jiang, J., Wolk, A. and Vessby, B. 1999. Am. J. Clin. Nutr. 70:21-27.
29. Belury, M. A. 2002. Annu. Rev. Nutr. 22:505-531.
30. Miller, A., McGrath, E., Stanton, C. and Devery, R. 2003. Lipids. 38:623-632.
31. Harfoot, C. G. and Hazlewood, G. P. 1997. Lipid metabolism in the rumen, p. 348-426. In P. N. Hobson and C. S. Stewart (ed), The rumen microbial ecosystem. Chapman & Hall, London.
32. Polan, C. E., McNeill, J. J. and Tove, S. B. 1964. J. Bacteriol. 88:1056-1064.
33. Howard, F. A. C. and Henderson, C. 1999. Letters in Appl. Microbiol. 29:193-196.
34. Edionwe, A. O., Kies, C. 2001. Plant Foods Hum Nutr. 56:157-65.
35. Kemp, M. Q., Jeffy, B. D. and Romagnolo, D. F. 2003. J. Nutr. 133:3670-3677.
36. Greicius, G., arulampalam, V. and Pettersson, S. 2004. Gastroenterol. 127:777-791.
37. Fukushima, M., Yamada, A., Endo, T. and Nakano, M. 1998. Nutrition. 15:373-378.
38. Mackay, F., J. L. Browning, P. Lawton, S. A. Shah, M. Comiskey, A. K. Bhan, E. Mizoguchi, C. Terhorst, and S. J. Simpson. Gastroenterology 115: 1464-1475, 1998.
39. Simopoulos, A. P. 2002. Biomed. Pharmacother. 56: 365-379.
40. Mills, S. C., A. C. Windsor, and S. C. Knight. 2005. Clin Exp Immun. 142: 216-228.
41. Bagga, D., L. Wang, R. Farias-Eisner, J. A. Glaspy, and S. T. Reddy. 2003. Proc. Natl. Acad. Sci. USA. 100: 1751-1756.
42. Robinson, J. G., and N. J. Stone. 2006. Am. J. Cardiol. 98: 39-49.
43. O'Hara A. M, and F. Shanahan. 2006. EMBO Rep. 7: 688-693.
44. Guarner, F. and J. R. Malagelada. 2003. Lancet. 361: 512-519.
45. Backhed, F., H. Ding, T. Wang, L. V. Hooper, G. Y. Koh, A. Nagy, C. F. Semenkovich, and J. I. Gordon. 2004. Proc. Natl. Acad. Sci. USA. 101: 15718-15723.
46. Kaplas, N., E. Isolauri, A. M. Lampi, T. Ojala, and K. Laitinen. 2007. Lipids. 45: 865-870.
47. Kankaanpää, P. E., B. Yang, H. P. Kallio, E. Isolauri, and S. J. Salminen. 2002. J. Nutr. Biochem. 13: 364-369.
48. Coakley, M., R. P. Ross, M. Nordgren, G. Fitzgerald, R. Devery, and C. Stanton. 2003. J. Appl. Microbiol. 94: 138-145.
49. Rosberg-Cody, E., R. P. Ross, S. Hussey, C. A. Ryan, B. P. Murphy, G. F. Fitzgerald, R. Devery, and C. Stanton. 2004. Appl. Environ. Microbiol. 70: 4635-4641.
50. Barrett, E., R. P. Ross, G. F. Fitzgerald, and C. Stanton. 2007. Appl. Environ. Microbiol. 73: 2333-2337.
51. Lee, K., K. Paek, H. Y. Lee, J. H. Park, and Y. Lee. 2007. J. Appl. Microbiol. 103: 1140-1146.
52. Yano, Y., A. Nakayama, H. Saito, and K. Ishihara. 1994. Lipids. 29: 527-528.
53. Yazawa K. 1996. Lipids. 31: S297-300.
54. Russell, N. J. and D. S, Nichols. 1999. Microbiology. 145: 767-779.
55. Metz, J. G., P. Roessler, D. Facciotti, C. Levering, F. Dittrich, M. Lassner, R. Valentine, K. Lardizabal, F. Domergue, A. Yamada, K. Yazawa, V. Knauf, and J. Browse. 2001. Science. 293: 290-293.
56. Orikasa, Y., A. Yamada, R. Yu, Y. Ito, T. Nishida, I. Yumoto, K. Watanabe, and H. Okuyama. 2004. Cell. Mol. Biol. 50: 625-63
57. O'Fallon, J. V., J. R. Busboom, M. L. Nelson, and C. T. Gaskins. 2007. J. Anim. Sci. 85: 1511-1521.
58. Favier, C., C. Neut, C. Mizon, A. Cortot, J. F. Colombel, and J. Mizon. 1997. Dig. Dis. Sci. 42: 817-822.
59. Swidsinski, A., A. Ladhoff, A. Pernthaler, S. Swidsinski, V. Loening-Baucke, M. Ortner, J. Weber, U. Hoffmann, S. Schreiber, M. Dietel, and H. Lochs. 2002. 122: 44-54.
60. Simopoulos, A. P. 2003. World. Rev. Nutr. Diet. 92: 1-22.
61. Wallace, J. L. 2001. Gastroenterol. Clin. North Am. 30: 971-980.
62. Simopoulos, A. P., A. Leaf, and N. Salem. 2000. Prostaglandins Leukot. Essent. Fatty Acids. 63: 119-121.
63. Jupp, J., K. Hillier, D. H. Elliott, D. R. Fine, A. C. Bateman, P. A. Johnson, A. M. Cazaly, J. F. Penrose, and A. P. Sampson. 2007. Inflamm. Bowel Dis. 13: 537-546.
64. Fu, Z. and A. J. Sinclair. 2000. Lipids. 35: 395-400.

1. (canceled)

2. The method of claim 19, wherein the CLA-producing bacterium is selected from one or more of the group consisting of propionibacteria, lactobacilli, lactococci and streptococci, and bifidobacteria.

3. The method of claim 2, wherein the CLA-producing bacterium is one or more of Bifidobacterium breve as deposited at the National Culture of Industrial and Marine Bacteria under the accession nos. 702258, B. breve DPC 6330 as deposited at the National Culture of Industrial and Marine Bacteria under the accession no. 41497 on 28 Sep. 2007, or B. longum DPC 6315 as deposited at the National Culture of Industrial and Marine Bacteria under the accession no. 41508 on 18 Oct. 2007.

4. (canceled)

5. The method of claim 20, wherein the CLA-producing bacterium is selected from one or more of the group consisting of propionibacteria, lactobacilli, lactococci and streptococci, and bifidobacteria.

6. The method of claim 5, wherein the CLA-producing bacterium is one or more of Bifidobacterium breve as deposited at the National Culture of Industrial and Marine Bacteria under the accession nos. 702258, B. breve DPC 6330 as deposited at the National Culture of Industrial and Marine Bacteria under the accession no. 41497 on 28^{th} Sep. 2007, or B. longum DPC 6315 as deposited at the National Culture of Industrial and Marine Bacteria under the accession no. 41508 on 18^{th} Oct. 2007.

7. The method of claim 20, wherein the organ is the liver.

8. A method of treating inflammatory disease in a subject, comprising administering to the subject a CLA-producing bacterium.

9. The method of claim 8, wherein the CLA-producing bacterium is selected from one or more of the group consisting of propionibacteria, lactobacilli, lactococci and streptococci, and bifidobacteria.

10. The method of claim 8 or 9 wherein the CLA-producing bacterium is one or more of Bifidobacterium breve as deposited at the National Culture of Industrial and Marine Bacteria under the accession nos. 702258, B. breve DPC 6330 as deposited at the National Culture of Industrial and Marine Bacteria under the accession no. 41497 on 28^{th} Sep. 2007, or B. longum DPC 6315 as deposited at the National Culture of Industrial and Marine Bacteria under the accession no. 41508 on 18 Oct. 2007.

11. A composition comprising a CLA producing organism together with pharmaceutically acceptable or nutritionally acceptable additives.

12. A composition as claimed in claim 11 wherein the CLA-producing bacterium is selected from one or more of the group consisting of propionibacteria, lactobacilli, lactococci and streptococci, and bifidobacteria.

13. A composition as claimed in claim 11 or 12 wherein the CLA-producing bacterium is one or more of Bifidobacterium breve as deposited at the National Culture of Industrial and Marine Bacteria under the accession nos. 702258, B. breve DPC 6330 as deposited at the National Culture of Industrial and Marine Bacteria under the accession no. 41497 on 28 Sep. 2007, or B. longum DPC 6315 as deposited at the National Culture of Industrial and Marine Bacteria under the accession no. 41508 on 18 Oct. 2007.

14. A probiotic composition as claimed in claim 11 or 12 which is a foodstuff selected from a yoghurt, a yoghurt drink, a cheese, a milk, a spread, a fruit juice, a water which is either flavoured or unflavoured.

15. A probiotic composition as claimed in claim 11 or 12 further comprising a substrate, which can be converted into a bioactive compound in vivo by the CLA producing organism.

16. A probiotic composition as claimed in claim 15 when the substrate is a polyunsaturated fatty acid.

17. A probiotic composition as claimed in claim 16 wherein the polyunsaturated fatty acid is linoleic acid, linolenic acid, oleic acid, palmitic acid or stearic acid.

18. A probiotic composition as claimed in claim 11 or 12 which can reduce gut inflammation in diseases such as Inflammatory Bowel Syndrome or Inflammatory Bowel Disease, rheumatoid arthritis, multiple sclerosis, Alzheimer's disease, eczema, asthma or psychiatric diseases such as depression.

19. A method of converting dietary polyunsaturated fatty acids to CLA in vivo comprising administration to a subject a live CLA-producing bacterial strain.

20. A method of altering the fatty acid composition of internal organs of the body comprising administering to a subject a live CLA-producing bacterial strain.

21. A method to drive DHA (docosahexaenoic acid)/EPA (eicosapentaenoic acid) incorporation into host tissues using dietary CLA or a strain producing CLA, in order to improve memory loss and cognition.

22. B. breve DPC 6330 as deposited at the National Culture of Industrial and Marine Bacteria under the accession no. 41497 on 28^{th} Sep. 2007.

23. B. longum DPC 6315 as deposited at the National Culture of Industrial and Marine Bacteria under the accession no. 41508 on 18^{th} Oct. 2007.

24. A feedstuff for animals comprising a CLA producing organism together with nutritionally acceptable ingredients.

25. A feedstuff as claimed in claim 24 further comprising a substrate which can be converted into a bioactive compound in vivo by the CLA producing organism.
